(19) United States
(12) Patent Application Publication
(10) Pub. No.: US 2021/0375399 A1
(43) Pub. Date: Dec. 2, 2021

(54) METHOD FOR ANALYSIS OF DRUG MOLECULAR DYNAMICS RESULTS BASED ON ROOT-MEAN-SQUARE DEVIATION MULTIPLE FEATURES

(71) Applicant: OCEAN UNIVERSITY OF CHINA, Qingdao (CN)

(72) Inventors: Hao LIU, Qingdao (CN); Zhiyu ZHANG, Qingdao (CN); Ximing XU, Qingdao (CN)

(73) Assignee: OCEAN UNIVERSITY OF CHINA, Qingdao (CN)

(21) Appl. No.: 17/315,335

(22) Filed: May 9, 2021

(30) Foreign Application Priority Data
May 26, 2020 (CN) 202010454509.9

(51) Int. Cl. G16C 10/00; G16B 5/00; G16B 15/00

(52) U.S. Cl. CPC G16C 10/00; G16B 15/00; G16B 5/00

(57) ABSTRACT

A method for an analysis of drug molecular dynamics results based on root-mean-square deviation multiple features is disclosed. The method includes the following steps: (i) analyzing and extracting features of a molecular dynamics RMSD image; (ii) performing a calculation of a binding free energy and an energy decomposition by using a molecular mechanics/Poisson-Boltzmann surface area method; and (iii) SVM classification training. By using RMSD image and molecular mechanics/Poisson-Boltzmann surface area method, the drug molecular dynamics are more efficient and accurate.

### CROSS REFERENCE TO THE RELATED APPLICATIONS

This application is based upon and claims priority to Chinese Patent Application No. 202010454509.9, filed on May 26, 2020, the entire contents of which are incorporated herein by reference.

### TECHNICAL FIELD

The present invention belongs to the technical field of drug molecular dynamics, and particularly relates to a method for an analysis of drug molecular dynamics results based on root-mean-square deviation (RMSD) multiple features.

### BACKGROUND

Molecular dynamics (MD) is a set of molecular simulation methods. The method mainly relies on Newtonian mechanics to simulate the motion of the molecular system to extract samples from systems composed of different states of the molecular system, thereby calculating the configuration integral of the system, and further calculating the thermodynamic quantities and other macroscopic properties based on the results of the configuration integral.

Molecular dynamics simulation has become a powerful tool and indispensable research means for studying molecular conformational changes and functional analysis, and has been widely used in drug design, life sciences, chemical engineering, physical sciences, drug research and development, materials science and other fields. This method can predict, guide and explain experiments to a large extent. The combination of computational simulations and experiments has become the main research method currently used.

The most important thing in medicinal chemistry is to have promising lead compounds, and one of the two pillars of sources for lead compounds is virtual screening technology. As the final step of the coupled technology of virtual screening, dynamics serves to select the most promising molecules for synthesis and improving the positive success rate of the lead compound of the target enzyme. In addition, dynamics can be used to study known compounds to further modify the compounds, and to explore more protein properties in the field of biochemistry, making it possible to dynamically design drug molecular lead compounds.

The calculation of binding free energy essentially deals with the change in energy of the system under investigation from one thermodynamic state to another thermodynamic state, either as a change in itself (valence change) or as a change in the surrounding environment (a change from vacuum to solvent, or a change from the solvent to the coordination environment), which can characterize the strength of the bonding from the perspective of total energy. In general, the more negative the binding free energy, the stronger the bonding, and the greater the energy required to break a bonding. In addition, if the binding free energy is positive, it indicates that the bonding cannot be formed spontaneously. The calculation of binding free energy not only evaluates the affinity between the ligand and the receptor determined experimentally, but also serves as a reference for drug design. So far, many methods for calculating the binding free energy have been widely used, mainly including the three categories as follows.

(1) Classical free energy calculation methods. Both the thermodynamic integration (TDI) method and the free energy perturbation (FEP) method are classical methods. The classical free energy calculation methods have a very strict theoretical basis and are suitable for almost any system, but they require substantial time to sample and the calculation amount is too large, so they can only be applied in simple cases.

(2) A method based on empirical equations. In this method, the binding free energy is decomposed into several energy terms, and then the empirical formula is obtained by using statistical methods with reference to a training set. This method has the advantages of short sampling time and minimal calculations, and disadvantages, such as an over-reliance on the training set, an inability to consider flexibility and solvation effects, and the method is not universally applicable.

(3) Empirical free energy calculation methods, which mainly include the linear interaction energy (LIE) method and molecular mechanics Poisson-Boltzmann (or generalized Born)/surface area (MM-PB/GBSA) method. The unique advantage of this type of method is that it can calculate the binding free energy of a ligand to a receptor.

Molecular mechanics/Poisson-Boltzmann surface area (MM/PBSA) method is used in biological macromolecular systems, including conformational changes of DNA, protein-protein, protein-DNA, and protein-small molecule interactions. In this method, the binding free energy is divided into a dynamic term, a solvation effect term, and an entropy change, which are calculated separately. Moreover, the dynamic term includes three terms Eᵢₙₜ, E_{edW} and E_{elec}, where int refers to bond, bond angle and dihedral angle. The solvation effect term is divided into polar term and non-polar term, which can be generally calculated with adaptive Poisson-Boltzmann solver (APBS) program. The entropy change is the most troublesome and the most inaccurate item. The existing calculation methods include the commonly used normal mode analysis method, and the quasi-harmonic analysis method.

Support vector machine (SVM) is a kind of generalized linear classifier for binary classification of data according to supervised learning, the decision boundary of which is the maximum margin hyperplane for solving learning samples. The SVM uses hinge loss function to calculate empirical risk and has added regularization term into the solving system to optimize structural risk, which is a classifier with sparsity and robustness. The SVM can conduct non-linear classification through kernel method, which is one of the common kernel learning methods.

Although the speed of the current molecular dynamics virtual screening method has been greatly improved, there are still many disadvantages.

1. In the analysis of molecular dynamics results, the method of calculating the binding free energy of compound conjugates typically used ignores the structural characteristics of the compound and cannot fully reflect the overall degree of binding between the target point and compound, which is one-sided to a certain extent.

2. Existing methods for evaluating and classifying results typically adopt manual methods based on accumulated experience, which has low processing efficiency and a certain degree of subjectivity and one-sidedness, resulting in substantial errors in results classification.

### SUMMARY

In view of the disadvantages of the prior art, the present invention provides a method for an analysis of drug molecular dynamics results based on RMSD multiple features, which makes drug molecular dynamics more efficient and accurate through RMSD image and molecular mechanics/Poisson-Boltzmann surface area method.

In order to solve the above technical problems, the technical solution adopted by the present invention is as follows.

A method for an analysis of drug molecular dynamics results based on RMSD multiple features, including the following steps:

1) analyzing and extracting features of a molecular dynamics RMSD image;

2) performing a calculation of a binding free energy and an energy decomposition by using a molecular mechanics/Poisson-Boltzmann surface area method; and

3) performing an SVM classification training.

Further, the specific operations of step 1) include: analyzing overall features of the RMSD image, calculating an RMSD value between a compound structure and an original structure in each frame; subsequently, calculating an average value and a variance of the RMSD in an entire molecular dynamics process as features; and subsequently, performing a fast Fourier transform on RMSD overall polyline, transforming an image in a time domain to a frequency domain, performing a spectrum analysis, and extracting coefficients of top-ranking low-frequency terms in the results as one of the features.

Further, the specific operations of step 2) include: turning molecular dynamics (MD) trajectories into a single file, setting the number of charges, preferably setting a dielectric constant of water as 80 and a dielectric constant of protein factor as 4, respectively calculating a molecular mechanical energy, a polar solvation energy and a non-polar solvation energy, and combining the molecular mechanical energy, the polar solvation energy and the non-polar solvation energy into a binding free energy of a compound; subsequently, decomposing the binding free energy, calculating an energy corresponding to each amino acid residue in the compound, then finding out residues corresponding to top-ranking free energies that contribute to the most to the total binding free energy, and then calculating a correlation of energy changes of amino acids at active sites of a positive compound.

Further, the specific operations of step 3) include: preprocessing the RMSD image to remove obviously invalid images produced by the interruption of the calculation process or other reasons to obtain a preprocessed RMSD image; subsequently, normalizing the preprocessed RMSD image, and dividing a data set into a training set and a testing set by sampling technology; and subsequently, labeling each image, wherein an image with a favorable dynamic result is labeled as 1, and an image with a less favorable dynamic result is labeled as −1.

Additionally, the method further includes: vectorizing the processed feature data including the overall average value and the variance of the RMSD image, the average value of the RMSD image in a stationary period, the coefficients of top ten low-frequency terms and residue similarity to form a feature vector; subsequently, using an Sigmoid function as a kernel function and selecting initial parameters C and r to train a classifier model with the training set, and testing a classification accuracy of the classifier model with the testing set, and then continuously adjusting the parameters to improve the accuracy.

Compared with the prior art, the present invention has the advantages as follows.

(1) The present invention provides a set of feature extraction solutions based on RMSD statistical data, which can comprehensively analyze various features of the results.

(2) In the present invention, the machine learning method is integrated, and the SVM method is used to classify and screen the dynamics results, which ensures the accuracy of the analysis of the drug molecular dynamics results.

(3) In the present invention, the screening efficiency of positive small molecules is improved while maintaining a high screening accuracy rate, and the analysis process of molecular dynamics results is optimized.

### BRIEF DESCRIPTION OF THE DRAWINGS

FIG. 1 is a diagram showing extracted features of an RMSD image; and

FIG. 2 is a diagram showing a network structure after putting the features into a classifier model.

### DETAILED DESCRIPTION OF THE EMBODIMENTS

The present invention is further described below with reference to specific embodiments of the present invention. The embodiments described are only a part of the embodiments of the present invention rather than all. All other embodiments derived based on the embodiments of the present invention by those of ordinary skill in the art without creative efforts shall be considered as falling within the protection scope of the present invention.

### Embodiment 1

A method for an analysis of drug molecular dynamics results based on RMSD multiple features. The method includes the steps as follows.

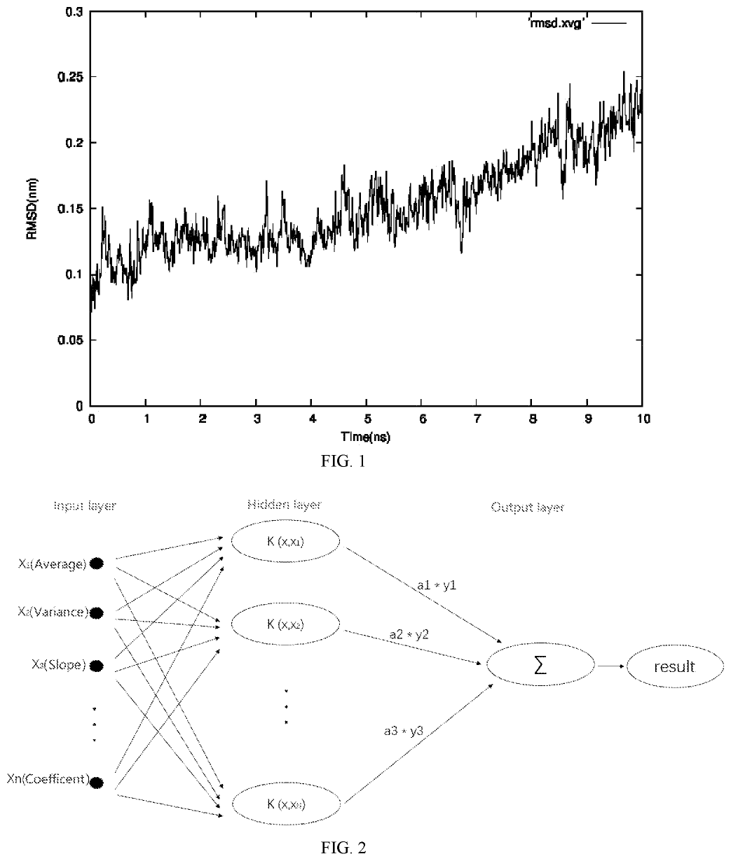

1) Analysis and extraction of features of molecular dynamics RMSD image Molecular dynamics simulation is carried out on 5V6A target (Middle East respiratory syndrome coronavirus protease, MERS-CoV) and drug molecular database DrugBank using molecular dynamics software, gnuplot is used to draw an RSMD change image (the RMSD image is shown in FIG. 1) during the dynamic process for the docking results, the overall features of the RMSD image are analyzed, and an RMSD value between a compound structure and an original structure in each frame is calculated. Subsequently, an average value (the average value as shown in FIG. 1 is 0.152026) and a variance (the variance as shown in FIG. 1 is 0.001309) of RMSD in the entire molecular dynamics process as features. Subsequently, the RMSD overall polyline is subjected to a fast Fourier transform, an image in a time domain is transformed to a frequency domain for a spectrum analysis, and coefficients of top-ranking low-frequency terms in the results (as shown in FIG. 1, the first five coefficients of the polynomial are 0.000018, −0.000508, 0.005309, −0.023626, 0.047714, respectively) are extracted as one of the features.

2) Calculation of binding free energy and energy decomposition by using molecular mechanics/Poisson-Boltzmann surface area method

The MD trajectories are turned into a single file, the number of charges is set, the dielectric constant of water is preferably set as 80, and the dielectric constant of protein factor is set as 4. A molecular mechanical energy, a polar solvation energy and a non-polar solvation energy are respectively calculated and combined into a binding free energy of a compound. Subsequently, the binding free energy is decomposed by MmPbSaDecomp.py script, an energy corresponding to each amino acid residue in the compound is calculated, and residues corresponding to top-ranking free energies that contribute the most to the total binding free energy (as shown in Table 1) are identified, and then a correlation of energy changes of the amino acids at active sites of a positive compound (the correlation is shown in Table 1) is calculated.

3) SVM Classification Training

The RMSD image is preprocessed, and then the RMSD image is normalized, and a data set is divided into a training set and a testing set by using sampling technology. Subsequently, each image is labeled, the images with a favorable dynamic result are labeled as 1, and the images with less favorable dynamic results are labeled as −1. Further, processed feature data such as the overall average value and the variance of the RMSD image, the average value of the RMSD image in a stationary period, the coefficients of top ten low-frequency terms and the residue similarity are vectorized to form a feature vector, the Sigmoid function is used as a kernel function, and initial parameters C and r are used to train a classifier model with the training set (the network structure diagram is shown in FIG. 2), a classification accuracy of the classifier model is tested with the testing set, and the parameters are continuously adjusted to improve the accuracy. Experiments show that the classification accuracy is about 80%. The experimental data of accuracy rates is shown in Table 2.

TABLE 1

| Residues/ | | | | | | | | | | | |
|---|---|---|---|---|---|---|---|---|---|---|---|
| kJ/mol | ID42581 | ID43391 | ID43392 | ID43393 | ID43395 | ID43397 | ID44486 | ID44620 | ID44664 | ID45732 | Yx |
| ARG-3 | −31.352 | −30.415 | −29.082 | −35.423 | −34.6255 | −33.051 | −25.088 | −27.637 | −35.385 | −36.378 | −37.6375 |
| LYS-6 | −29.314 | −31.280 | −24.259 | −35.259 | −33.258 | −28.265 | −39.254 | −42.215 | −30.255 | −29.258 | −32.5392 |
| ASP-22 | 39.2587 | 35.6854 | 48.2587 | 40.2598 | 38.2587 | 41.0258 | 48.2598 | 23.2587 | 35.0258 | 36.5874 | 37.9035 |
| ASP-40 | 32.0548 | 30.2598 | 42.2158 | 29.2587 | 38.2587 | 40.2587 | 46.2587 | 45.3687 | 39.0247 | 29.0248 | 34.2008 |

Calculation formula: Σ((residue energy − yx energy)/yx energy) × 25%

TABLE 2

| ID | Judgment result | Energy similarity | Correct/Error |
|---|---|---|---|
| 42581 | 1 | 91.25 | Correct |
| 43391 | 1 | 89.75 | Correct |
| 43392 | 1 | 78.25 | Error |
| 43393 | 1 | 91.25 | Correct |
| 43395 | 1 | 94.25 | Correct |
| 43397 | 0 | 88.00 | Error |
| 44486 | 0 | 75.25 | Correct |
| 44620 | 0 | 71.50 | Correct |
| 44664 | 1 | 91.50 | Correct |
| 45732 | 1 | 91.75 | Correct |

What is claimed is:

1. A method for an analysis of drug molecular dynamics (MD) results based on root-mean-square deviation (RMSD) multiple features, comprising the following steps:
(i) analyzing and extracting features of an MD RMSD image;
(ii) performing a calculation of a binding free energy and an energy decomposition by using a molecular mechanics/Poisson-Boltzmann surface area method; and
(iii) performing an SVM classification training; wherein
step (i) comprises the following operations: analyzing features of the MD RMSD image,
calculating a root-mean-square deviation value (abbreviated as RMSD) between a compound structure and an original structure in each frame of an MD process and producing an RMSD polyline from the RMSD of each frame; calculating an average value of the RMSD polyline and a variance of the RMSD polyline; and performing a fast Fourier transform on the RMSD polyline, transforming the RMSD polyline in a time domain to a frequency domain for a spectrum analysis, and extracting coefficients of top-ranking low-frequency terms in results;
step (ii) comprises the following operations: calculating a molecular mechanical energy, a polar solvation energy and a non-polar solvation energy, and combining the molecular mechanical energy, the polar solvation energy and the non-polar solvation energy into the binding free energy of the compound structure; decomposing the binding free energy, calculating an energy corresponding to each amino acid residue in the compound structure, then finding out residues corresponding to top-ranking free energies, wherein the top-ranking free energies contribute to the most to a total binding free energy, and then calculating a correlation of energy changes of amino acids at active sites of the compound structure;
step (iii) comprises the following operations: preprocessing the molecular dynamics RMSD image to remove obviously invalid images produced by an interruption of a calculation process or other reasons to obtain a preprocessed RMSD image; normalizing the preprocessed RMSD image to obtain a treated RMSD image, and dividing the treated RMSD image into a training set and a testing set by a sampling technology; and labeling each frame of the treated RMSD image, wherein an image with a favorable dynamic result is labeled as 1, and an image with a less favorable dynamic result is labeled as −1;
vectorizing processed feature data comprising the average value of the RMSD polyline and the variance of the RMSD polyline, an average value of the molecular dynamics RMSD image in a stationary period, the coefficients of top ten low-frequency terms and a residue similarity to form a feature vector; using an Sigmoid function as a kernel function and training a classifier model with the training set, and testing a classification accuracy of the classifier model with the testing set, and then continuously adjusting parameters to improve the classification accuracy.
